(19) United States
(12) Patent Application Publication
(10) Pub. No.: US 2026/0242380 A1
(43) Pub. Date: Aug. 20, 2026

(54) METHODS FOR PREPARING PYRROLIZIDINE COMPOUNDS

(71) Applicant: Frontier Medicines Corporation, South San Francisco, CA (US)

(72) Inventors: Jacob Y. CHA, South San Francisco, CA (US); Peter LIN, South Plainfield, NJ (US)

(21) Appl. No.: 19/161,118

(22) PCT Filed: Mar. 1, 2024
(86) PCT No.: PCT/US2024/018219

§ 371 Date: Aug. 29, 2025

Related U.S. Application Data
(60) Provisional application No. 63/488,356, filed on Mar. 3, 2023.

(51) Int. Cl. C07D 487/04; C07D 487/10

(52) U.S. Cl. CPC C07D 487/04; C07D 487/10

(57) ABSTRACT

Provided herein, in part, is a method of preparing a compound of Formula (II-1): or a salt thereof, wherein the method comprises reacting a compound of Formula (III-1): with a first base in the presence of a first solvent, thereby producing a compound of Formula (II-1) or a salt thereof, wherein R is an ester protecting group and X is a counter ion.

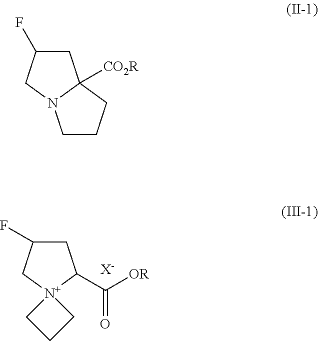

### CROSS-REFERENCE TO RELATED APPLICATION

This application claims priority to and benefit of U.S. Provisional Patent Application No. 63/488,356, filed Mar. 3, 2023, the disclosure of which is hereby incorporated herein by reference in its entirety.

### FIELD OF THE DISCLOSURE

The present disclosure provides compounds and synthetic methods for preparing compounds that may be used for preparing compounds useful in treating or suppressing cancer, and in particular, useful in treating or suppressing cancers.

### BACKGROUND

KRAS is a molecular switch. Under normal physiological conditions, the protein is bound to guanosine diphosphate (GDP) in the “off state.” In response to signaling through receptor tyrosine kinases (RTKs) such as EGFR, the GDP is exchanged to guanosine triphosphate (GTP) in a process facilitated by guanine nucleotide exchange factors (GEFs) such as SOS. The GTP-bound form of KRAS is in the “on state,” and interacts with proteins such as RAF and PI3K to promote downstream signaling that leads to cell proliferation and survival. KRAS can slowly hydrolyze GTP back to GDP, thus returning to the off-state, in a process facilitated by GAPs (GTPase-activating Proteins).

KRAS mutations are found in approximately 30% of all human cancers, and are highly prevalent among three of the deadliest forms of cancer: pancreatic (95%), colorectal (45%), and lung (35%). Together, these cancers occur in more than 200,000 patients annually in the US alone. One particular mutation, a glycine to cysteine substitution at position 12 (G12C), occurs in more than 40,000 patients per year. The KRAS G12C mutation impairs hydrolysis of GTP to GDP, thus trapping KRAS in the on-state and promoting cancer cell proliferation.

### SUMMARY

Provided herein in part is a method of preparing a compound of Formula (II-1):

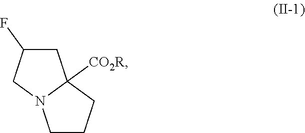

or a salt thereof, wherein the method comprises reacting a compound of Formula (III-1):

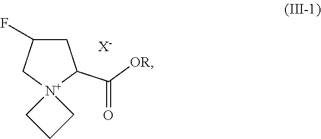

with a first base in the presence of a first solvent, thereby producing a compound of Formula (II-1) or a salt thereof, wherein R is an ester protecting group and X is a counter ion.

In some embodiments, the compound of Formula (II-1) is a compound of Formula (IIa-1):

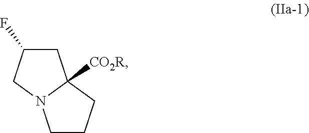

or a salt thereof, and the compound of Formula (III-1) is a compound of Formula (IIIa-1):

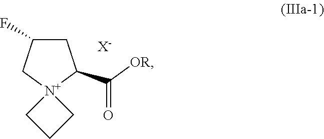

In some embodiments, the method further comprises: reacting a compound of Formula (IV-1):

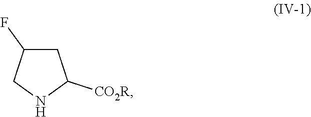

or a salt thereof, with an alkylating agent in the presence of a second base and a second solvent, thereby producing a compound of Formula (III-1):

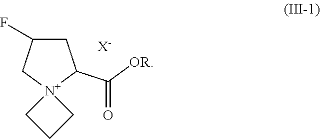

In some embodiments, the compound of formula (IV-1) is a compound of Formula (IVa-1):

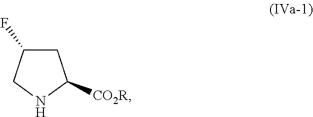

or a salt thereof.

In some embodiments, the method further comprises reacting a compound of Formula (V-1):

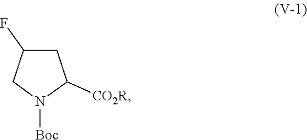

or a salt thereof, with an acid in the presence of a third solvent, thereby producing a compound of Formula (IV-1):

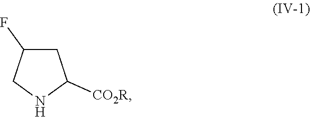

or a salt thereof.

In some embodiments, the compound of Formula (V-1) is a compound of Formula (Va-1):

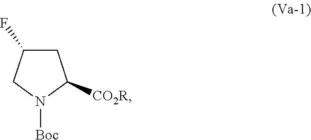

or a salt thereof.

In some embodiments, the method further comprises reacting a compound of Formula

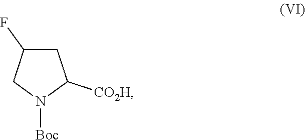 [SMILES: CC(C)(C)OC(=O)N1CC(F)CC1C(=O)O]

or a salt thereof, with an agent that introduces an R group in the presence of a third base and a fourth solvent, thereby producing a compound of Formula (V-1):

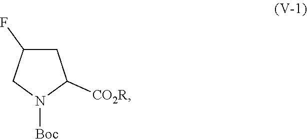

or a salt thereof.

In some embodiments, the compound of Formula (VI) is a compound of Formula (VIa):

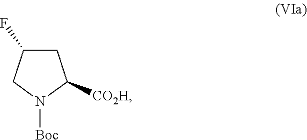 [SMILES: CC(C)(C)OC(=O)N1C[C@H](F)C[C@H]1C(=O)O]

or a salt thereof.

Also provided herein is a method of preparing a compound of Formula (I):

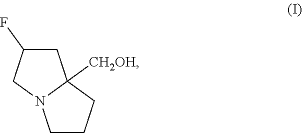 [SMILES: OCC12CCCN1CC(F)C2]

or a salt thereof, wherein the method comprises:
reacting a compound of Formula (II-1):

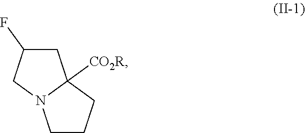

or a salt thereof with a reducing agent in the presence of a fifth solvent, thereby producing a compound of Formula (I) or a salt thereof, wherein the compound of Formula (II-1) or a salt thereof is prepared according to a method described herein.

In some embodiments, the compound of Formula (I) is a compound of Formula (Ia):

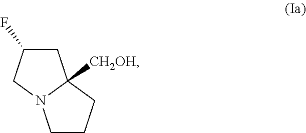 [SMILES: OC[C@@]12CCCN1C[C@H](F)C2]

or a salt thereof.

The present disclosure also provides a method of preparing a compound of Formula (I):

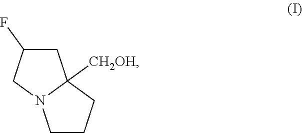 [SMILES: OCC12CCCN1CC(F)C2]

or a salt thereof, wherein the method comprises the steps of:
(1) reacting a compound of formula (VI):

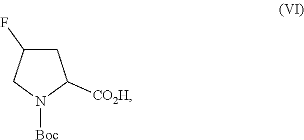 [SMILES: CC(C)(C)OC(=O)N1CC(F)CC1C(=O)O]

or a salt thereof with an agent that introduces an R group, thereby producing a compound of Formula (V-1):

or a salt thereof;

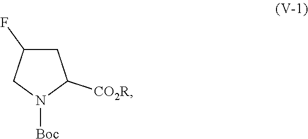

(2) reacting a compound of Formula (V-1) or a salt thereof with an acid, thereby producing a compound of Formula (IV-1):

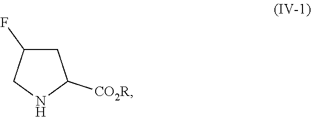

or a salt thereof;
(3) reacting a compound of Formula (IV-1) or a salt thereof with an alkylating agent, thereby producing a compound of Formula (III-1):

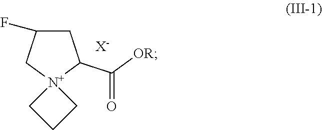

(4) reacting a compound of Formula (III-1) with a base, thereby producing a compound of Formula (II-1):

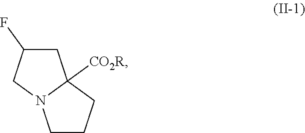

or a salt thereof; and
(5) reacting a compound of Formula (II-1) or a salt thereof with a reducing agent, thereby producing a compound of Formula (I) or a salt thereof;
   wherein R is an ester protecting group and X is a counter ion.

In another aspect, the present disclosure provides a compound of Formula (III):

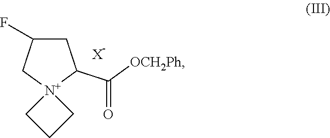

wherein X⁻ is a counter ion.

In some embodiments, the compound is a compound of Formula (IIIa):

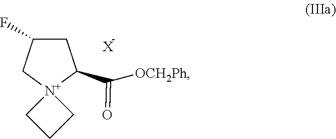

wherein X⁻ is a counter ion.

In another aspect, the present disclosure provides a compound of Formula (II):

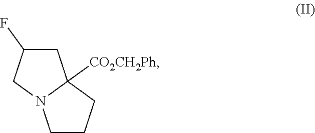

or a salt thereof.

In some embodiments, the compound is a compound of Formula (IIa):

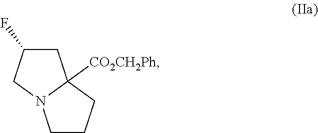

or a salt thereof.

In another aspect, the present disclosure provides a compound of Formula (II-1):

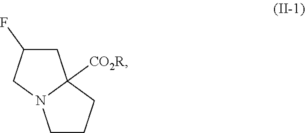

or a salt thereof, wherein R is an ester protecting group, wherein the compound is prepared according to a method described herein.

In another aspect, the present disclosure provides a compound of Formula (I):

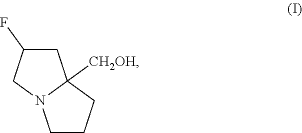 [SMILES: OCC12CCCN1CC(F)C2]

or a salt thereof, wherein the compound of Formula (I) is prepared according to a method described herein.

### DETAILED DESCRIPTION

Provided herein, in part, are methods for preparing compounds (e.g., synthetic intermediates) that may be used for preparing compounds useful for treating cancer. The contemplated methods may result in a higher stereoselectivity and/or yield of target compounds and therefore are more suitable for large scale manufacturing processes as compared to known methods. The present disclosure also provides novel compounds and compositions that may be used for preparing compounds useful for treating cancer. The cancer may be a cancer characterized by e.g., KRAS G12C.

In some embodiments, the compounds and synthetic methods described in the present disclosure may be used to prepare the compounds described in PCT application No. PCT/US2022/079324, the contents of which is hereby incorporated by reference.

The abbreviations used herein have their conventional meaning within the chemical and biological arts, unless otherwise specified.

It is to be understood that descriptions of compound structures, including possible substitutions, are limited to those which are chemically possible.

Unless otherwise indicated, the absolute stereochemistry of all chiral atoms is as depicted. A person of skill in the art would be able to separate racemic compounds into the respective enantiomers using methods known in the art, such as chiral chromatography, chiral recrystallization and the like. References to compounds that are racemic mixtures are meant to also include the individual enantiomers contained in the mixture.

Reference to “about” a value or parameter herein includes (and describes) variations that are directed to that value or parameter per se. For example, description referring to “about X” includes description of “X”. Specifically, the terms “about” contemplate a value within 15%, within 10%, within 5%, within 4%, within 3%, within 2%, within 1%, or within 0.5% of the specified value.

As used herein, and unless otherwise specified, the terms “about” when used in connection with temperatures, mean a temperature that is recognized by those of ordinary skill in the art to provide a chemical reaction outcome equivalent to that obtained from the specified temperature.

The terms “a” and “an,” as used in herein mean one or more, unless context clearly dictates otherwise.

While the compounds described herein can occur and can be used as the neutral (non-salt) compound, the description is intended to embrace all salts of the compounds described herein, as well as methods of using such salts of the compounds. In some embodiments, the salts of the compounds comprise pharmaceutically acceptable salts.

The term “pharmaceutically acceptable salt” of a compound means a salt that is pharmaceutically acceptable to humans and/or animals, and which, upon administration, retains at least some of the desired pharmacological activity of the parent compound. Such salts include: (a) acid addition salts, formed with inorganic acids such as hydrochloric acid, hydrobromic acid, sulfuric acid, nitric acid, phosphoric acid, and the like; or formed with organic acids such as formic acid, acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (b) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. Additional information on suitable pharmaceutically acceptable salts can be found in Remington's Pharmaceutical Sciences, 17th ed., Mack Publishing Company, Easton, PA, 1985, which is incorporated herein by reference in its entirety.

Included herein, when chemically relevant, are all stereoisomers of the compounds, including diastereomers and enantiomers. Also included are mixtures of possible stereoisomers in any ratio, including, but not limited to, racemic mixtures. Unless stereochemistry is explicitly indicated in a structure, the structure is intended to embrace all possible stereoisomers of the compound depicted. If stereochemistry is explicitly indicated for one portion or portions of a molecule, but not for another portion or portions of a molecule, the structure is intended to embrace all possible stereoisomers for the portion or portions where stereochemistry is not explicitly indicated.

The term “alkylating agent” as used herein refers to a chemical agent that introduces an alkyl or alkylene group.

The term “alkyl” refers to a linear, branched, cyclic, or a combination thereof, saturated monovalent hydrocarbon radical having the defined number of carbons. For example, alkyl may be C₁-C₆ alkyl, which includes e.g., methyl, ethyl, propyl, 2-propyl, butyl, cyclopropyl, cyclobutyl, pentyl, hexyl, and the like.

The term “alkylene” means a linear, branched, cyclic, or a combination thereof, saturated divalent hydrocarbon radical having the defined number of carbons. For example, alkylene may be C₁-C₆ alkylene, which includes e.g., methylene, ethylene, propylene, 1-methylpropylene, 2-methylpropylene, butylene, pentylene, hexylene and the like.

The term “metal alkoxide” as used herein refers to a compound derived from replacement of the hydroxy hydrogen with a metal. In some embodiments, the metal alkoxide may be potassium t-butoxide.

The term “metal hydride” as used herein refers to a compound containing a metal bonded to hydrogen.

The term “counter ion” as used herein refers to an ion that accompanies an ionic compound to maintain electronic neutrality.

The term “ester protecting group” as used herein refers to a functional group that protects a carboxylic acid by replacing the acidic proton of the carboxylic acid to form an ester. For example, the ester protecting group may be an alkyl group or benzyl group. A benzyl group is a univalent radical —CH₂—C₆H₅.

The term “polar aprotic solvent” as used herein refers to a polar solvent that does not have an acidic proton.

A compound that is “diastereomerically pure” is substantially free of another diastereomer. In some embodiments, the compound may be >95% pure, >97% pure, >98% pure, or >99% pure as measured by HPLC or NMR. In other embodiments, the diastereomerically pure compound is obtained as a single diastereomer and no other diastereomer is detected by HPLC or NMR.

The term “halo” refers to fluoro, chloro, bromo, or iodo.

The term “benzyl halide” refers to benzyl fluoride, benzyl chloride, benzyl bromide, or benzyl iodide.

The term “room temperature” as used herein refers to a temperature of about 20-25° C.

### Selected Embodiments

Embodiment 1. A method of preparing a compound of Formula (II-1):

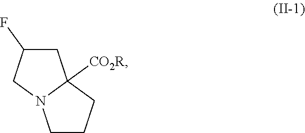

or a salt thereof, wherein the method comprises reacting a compound of Formula (III-1):

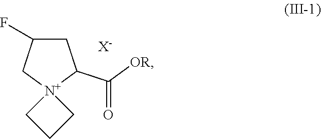

with a first base in the presence of a first solvent, thereby producing a compound of Formula (II-1) or a salt thereof, wherein R is an ester protecting group and X is a counter ion.
Embodiment 2. The method of embodiment 1, wherein the reaction of a compound of formula (III-1) with a first base is conducted at between about 0° C. to about room temperature.
Embodiment 3. The method of embodiment 1 or 2, wherein the first base is a metal alkoxide base.
Embodiment 4. The method of embodiment 3, wherein the first base is KOtBu.
Embodiment 5. The method of any one of embodiments 1-4, wherein the first solvent is a polar aprotic solvent.
Embodiment 6. The method of embodiment 5, wherein the polar aprotic solvent is an ethereal solvent.
Embodiment 7. The method of embodiment 5 or 6, wherein the polar aprotic solvent is THF or 2-MeTHF.
Embodiment 8. The method of any one of embodiments 1-7, wherein the method produces a compound of formula (II-1) as a diastereomerically pure compound.
Embodiment 9. The method of any one of embodiments 1-8, wherein the compound of Formula (II-1) is a compound of Formula (IIa-1):

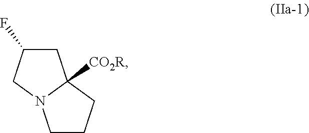

or a salt thereof, and the compound of Formula (III-1) is a compound of Formula(IIIa-1):

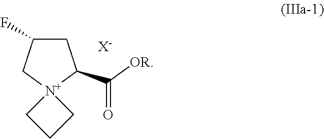

Embodiment 10. The method of any one of embodiments 1-9, wherein the method further comprises: reacting a compound of Formula (IV-1):

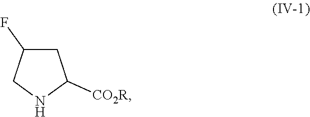

or a salt thereof, with an alkylating agent in the presence of a second base and a second solvent, thereby producing a compound of Formula (III-1):

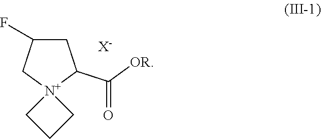

Embodiment 11. The method of embodiment 10, wherein the reaction of a compound of formula (IV-1) with an alkylating agent is conducted at about room temperature.
Embodiment 12. The method of any one of embodiments 1-11, wherein X⁻ is triflate (TfO⁻) ion.
Embodiment 13. The method of any one of embodiments 10-12, wherein the alkylating agent is

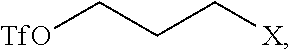

wherein X is triflate or halide.
Embodiment 14. The method of embodiment 13, wherein the alkylating agent is:

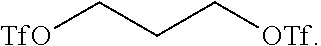 [SMILES: O=S(=O)(OCCCOS(=O)(=O)C(F)(F)F)C(F)(F)F]

Embodiment 15. The method of any one of embodiments 10-14, wherein the second base is an amine.
Embodiment 16. The method of any one of embodiments 10-14, wherein the second base is a weak base anion-exchange resin.
Embodiment 17. The method of embodiment 16, wherein the anion-exchange resin is Amberlyst™ A-21.
Embodiment 18. The method of any one of embodiments 15-17, wherein the amine is N,N-diisopropylethylamine.
Embodiment 19. The method of any one of embodiments 10-18, wherein the second solvent is a nonpolar solvent.
Embodiment 20. The method of any one of embodiments 10-19, wherein the second solvent is selected from the group consisting of toluene, THF, 2-MeTHF, and acetonitrile.
Embodiment 21. The method of embodiment 20, wherein the second solvent is acetonitrile.
Embodiment 22. The method of any one of embodiments 10-21, wherein the compound of formula (IV-1) is a compound of Formula (IVa-1):

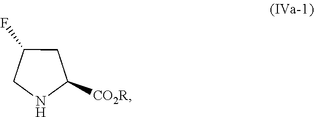

or a salt thereof.
Embodiment 23. The method of any one of embodiments 10-22, wherein the method further comprises reacting a compound of Formula (V-1):

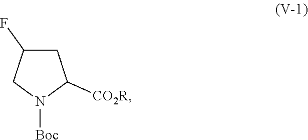

or a salt thereof, with an acid in the presence of a third solvent, thereby producing a compound of Formula (IV-1):

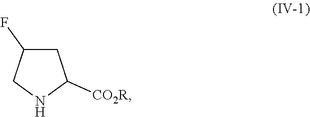

or a salt thereof.
Embodiment 24. The method of embodiment 23, wherein the acid is an organic acid or mineral acid.
Embodiment 25. The method of embodiment 24, wherein the acid is trifluoroacetic acid.
Embodiment 26. The method of any one of embodiments 23-25, wherein the third solvent is a polar aprotic solvent.
Embodiment 27. The method of any one of embodiments 23-26, wherein the third solvent is dichloromethane.
Embodiment 28. The method of any one of embodiments 23-27, wherein the compound of Formula (V-1) is a compound of Formula (Va-1):

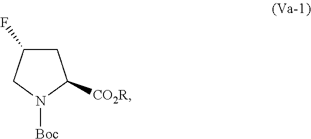

or a salt thereof.
Embodiment 29. The method of any one of embodiments 23-28, wherein the method further comprises reacting a compound of Formula (VI):

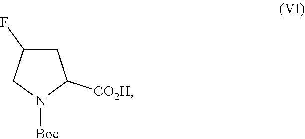 [SMILES: CC(C)(C)OC(=O)N1CC(F)CC1C(=O)O]

or a salt thereof, with an agent that introduces an R group in the presence of a third base and a fourth solvent, thereby producing a compound of Formula (V-1):

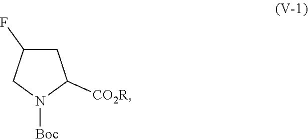

or a salt thereof.
Embodiment 30. The method of embodiment 29, wherein the third base is an amine base.
Embodiment 31. The method of embodiment 30, wherein the amine base is selected from the group consisting of N,N-diisopropylethylamine, triethylamine, and 1,8-diazabicyclo(5.4.0)undec-7-ene (DBU).
Embodiment 32. The method of any one of embodiments 29-31, wherein the fourth solvent is a polar aprotic solvent.
Embodiment 33. The method of embodiment 32, wherein the fourth solvent is acetonitrile.
Embodiment 34. The method of any one of embodiments 29-33, wherein the compound of Formula (VI) is a compound of Formula (VIa):

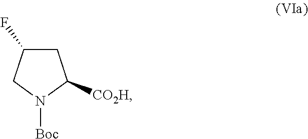 [SMILES: CC(C)(C)OC(=O)N1C[C@H](F)C[C@H]1C(=O)O]

or a salt thereof.
Embodiment 35. The method of any one of embodiments 29-34, wherein the agent that introduces an R group is a benzyl halide.
Embodiment 36. The method of embodiment 35, wherein the agent that introduces an R group is benzyl bromide.
Embodiment 37. The method of any one of embodiments 1-36, wherein R is benzyl.
Embodiment 38. A method of preparing a compound of Formula (I):

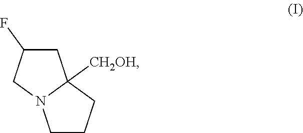 [SMILES: OCC12CCCN1CC(F)C2]

or a salt thereof, wherein the method comprises:
reacting a compound of Formula (II-1):

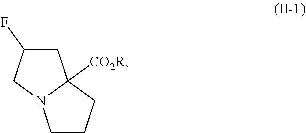

or a salt thereof with a reducing agent in the presence of a fifth solvent, thereby producing a compound of Formula (I) or a salt thereof, wherein the compound of Formula (II-1) or a salt thereof is prepared according to a method of any one of embodiments 1-37.
Embodiment 39. The method of embodiment 38, wherein the reducing agent is a metal hydride.
Embodiment 40. The method of embodiment 39, wherein the metal hydride is selected from the group consisting of LiAlH₄, LiBH₄, and Red-Al®.
Embodiment 41. The method of any one of embodiments 38-40, wherein the fifth solvent is a polar aprotic solvent.
Embodiment 42. The method of any one of embodiments 38-41, wherein the fifth solvent is an ethereal solvent.
Embodiment 43. The method of any one of embodiments 38-42, wherein the fifth solvent is selected from the group consisting of THF, Me-THF, diethyl ether, dimethoxy ethane, and 1,4-dioxane.
Embodiment 44. The method of any one of embodiments 38-43, wherein the method produces a compound of formula (I) as a diastereomerically pure compound.
Embodiment 45. The method of any one of embodiments 38-44, wherein the compound of Formula (I) is a compound of Formula (Ia):

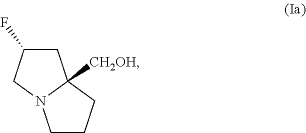 [SMILES: OC[C@@]12CCCN1C[C@H](F)C2]

or a salt thereof.
Embodiment 46. A method of preparing a compound of Formula (I):

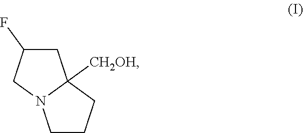 [SMILES: OCC12CCCN1CC(F)C2]

or a salt thereof, wherein the method comprises the steps of:
(1) reacting a compound of formula (VI):

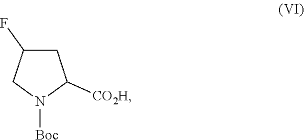 [SMILES: CC(C)(C)OC(=O)N1CC(F)CC1C(=O)O]

or a salt thereof with an agent that introduces an R group, thereby producing a compound of Formula (V-1):

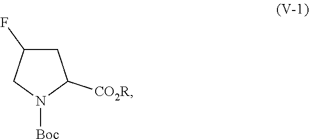

or a salt thereof;
(2) reacting a compound of Formula (V-1) or a salt thereof with an acid, thereby producing

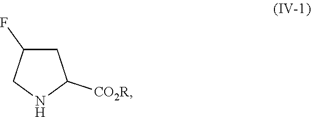

or a salt thereof;
(3) reacting a compound of Formula (IV-1) or a salt thereof with an alkylating agent, thereby producing a compound of Formula (III-1):

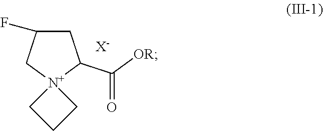

(4) reacting a compound of Formula (III-1) with a base, thereby producing a compound of Formula (II-1):

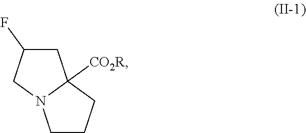

or a salt thereof; and
(5) reacting a compound of Formula (II-1) or a salt thereof with a reducing agent, thereby producing a compound of Formula (I) or a salt thereof;
   wherein R is an ester protecting group and X is a counter ion.
Embodiment 47. The method of embodiment 46, wherein step (4) produces a compound of formula (II-1) as a diastereomerically pure compound.
Embodiment 48. The method of embodiment 46 or 47, wherein the compound of Formula (II-1) is a compound of Formula (IIa-1):

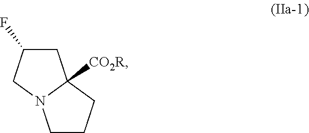

or a salt thereof, and the compound of Formula (III-1) is a compound of Formula (IIIa-1):

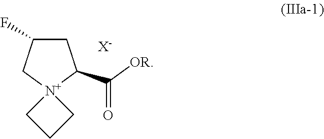

Embodiment 49. The method of any one of embodiments 46-48, wherein the compound of Formula (I) is a compound of Formula (Ia):

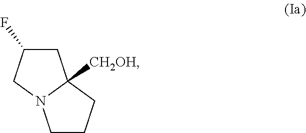 [SMILES: OC[C@@]12CCCN1C[C@H](F)C2]

or a salt thereof.
Embodiment 50. The method of any one of embodiments 46-49, wherein the compound of formula (IV-1) is a compound of Formula (IVa-1):

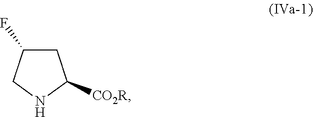

or a salt thereof.
Embodiment 51. The method of any one of embodiments 46-50, wherein the compound of Formula (V-1) is a compound of Formula (Va-1):

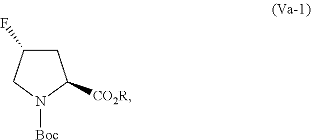

or a salt thereof.
Embodiment 52. The method of any one of embodiments 46-51, wherein the compound of Formula (VI) is a compound of Formula (VIa):

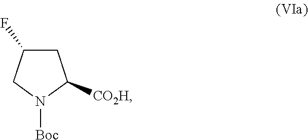 [SMILES: CC(C)(C)OC(=O)N1C[C@H](F)C[C@H]1C(=O)O]

or a salt thereof.
Embodiment 53. The method of any one of embodiments 46-52, wherein the reaction of step (4) is conducted at about 0° C. to about room temperature.
Embodiment 54. The method of any one of embodiments 46-53, wherein the reducing agent is a metal hydride.
Embodiment 55. The method of embodiment 54, wherein the metal hydride is selected from the group consisting of LiAlH₄, LiBH₄, or Red-Al®.
Embodiment 56. The method of embodiment 54, wherein the metal hydride is LiAlH₄.
Embodiment 57. The method of any one of embodiments 46-56, wherein the base is a metal alkoxide base.
Embodiment 58. The method of any one of embodiments 46-57, wherein the base is KO^{t}Bu.
Embodiment 59. The method of any one of embodiments 46-58, wherein the reaction of step (3) is conducted at room temperature.
Embodiment 60. The method of any one of embodiments 46-59, wherein X⁻ is triflate (TfO⁻) ion.
Embodiment 61. The method of any one of embodiments 46-60, wherein the alkylating agent is

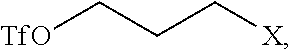

wherein X is triflate or halo.
Embodiment 62. The method of embodiment 61, wherein the alkylating agent is:

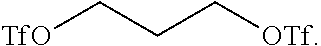 [SMILES: O=S(=O)(OCCCOS(=O)(=O)C(F)(F)F)C(F)(F)F]

Embodiment 63. The method of any one of embodiments 46-62, wherein the acid is an organic acid or mineral acid.
Embodiment 64. The method of embodiment 63, wherein the acid is trifluoroacetic acid.
Embodiment 65. The method of any one of embodiments 46-64, wherein R is benzyl.
Embodiment 66. The method of any one of embodiments 46-65, wherein the agent that introduces an R group is a benzyl halide.
Embodiment 67. The method of embodiment 66, wherein the agent that introduces an R group is benzyl bromide.
Embodiment 68. A compound of Formula (III):

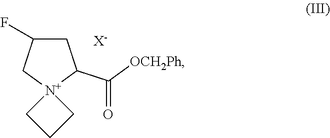

wherein X⁻ is a counter ion.
Embodiment 69. The compound of embodiment 68, wherein the compound is a compound of Formula (IIIa):

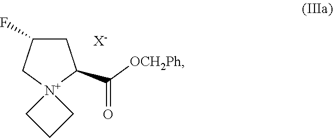

wherein X⁻ is a counter ion.
Embodiment 70. The compound of embodiment 68 or 69, wherein X⁻ is triflate (TfO⁻) ion.
Embodiment 71. A compound of Formula (II):

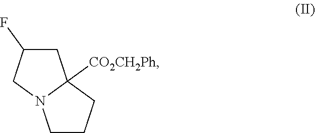

or a salt thereof.
Embodiment 72. The compound of embodiment 71, wherein the compound is a compound of Formula (IIa):

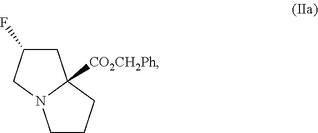

or a salt thereof.
Embodiment 73. A compound of Formula (II-1):

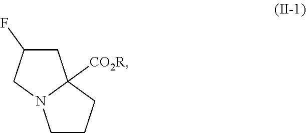

or a salt thereof, wherein R is an ester protecting group, wherein the compound is prepared according to a method of any one of embodiments 1-37.
Embodiment 74. A compound of Formula (I):

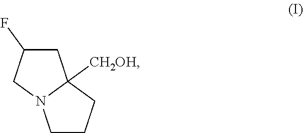 [SMILES: OCC12CCCN1CC(F)C2]

or a salt thereof, wherein the compound of Formula (I) is prepared according to a method of any one of embodiments 38-67.

### Synthetic Methods

Compounds of this disclosure can be made in view of the disclosure in the Examples shown below.

The starting materials and reagents used in preparing these compounds are either available from commercial suppliers such as MilliporeSigma., Bachem, etc. or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers. 1989): Organic Reactions, Volumes 1-40 (John Wiley and Sons, 1991), March's Advanced Organic Chemistry. (John Wiley and Sons, 4th Edition) and Larock's Comprehensive Organic Transformations (VCH Publishers Inc., 1989). These schemes are merely illustrative of some methods by which the compounds of this disclosure can be synthesized, and various modifications to these schemes can be made and will be suggested to one skilled in the art reading this disclosure. The starting materials and the intermediates, and the final products of the reaction may be isolated and purified if desired using conventional techniques, including but not limited to filtration, distillation, crystallization, chromatography and the like. Such materials may be characterized using conventional means, including physical constants and spectral data.

Unless specified to the contrary, the reactions described herein take place at atmospheric pressure over a temperature range from about 78° C. to about 150° C., such as from about 0° C. to about 125° C. and further such as at about room (or ambient) temperature, which is about 20° C.

### Examples

The following preparations of the compounds of formulae (I)-(VI) and salts thereof are given to enable those skilled in the art to more clearly understand and to practice the present disclosure. hey should not be considered as limiting the scope of the disclosure, but merely as being illustrative and representative thereof.

The following abbreviations are used in this section:

| | |
|---|---|
| ABq | AB quartet |
| Boc | tert-butyloxycarbonyl |
| br | Broad |
| CDCl₃ | Deuterated chloroform |
| d | Doublet |
| ddd | Doublet of doublet of doublet |
| dt | Doublet of triplet |
| dq | Doublet of quartet |
| DCM | dichloromethane |
| EtOAc | Ethyl acetate |
| h | Hour(s) |
| hex | Hexanes |
| HPLC | High performance liquid chromatography |
| iPr₂Net | N,N,-diisopropylethylamine (Hunig's base) |
| KOtBu | Potassium tert-butoxide |
| LCAP | Liquid chromatography area percent |
| LCMS | Liquid chromatography-mass spectrometry |
| LiAlH₄ | Lithium aluminum hydride |
| MeCN | Acetonotrile |
| MeOH | Methanol |
| m/z | Mass to charge ratio |
| MHz | Megahertz |
| mL | Mililiter(s) |
| min | Minute(s) |
| MTBE | tert-Butyl methyl ether |
| Na₂CO₃ | Sodium carbonate |
| NaOH | Sodium hydroxide |
| Na₂SO₄ | Sodium sulfate |
| NMR | Nuclear magnetic resonance |
| OTf | trifluoromethanesulfonate |
| Ph | Phenyl |
| q | quartet |
| RT | Room temperature |
| Rt | Retention time |
| s | Singlet |
| t | Triplet |
| tBu | t-butyl |
| TFA | Trifluoroacetic acid |
| Tf₂O | Trifluoromethanesulfonic anhydride |
| THF | Tetrahydrofuran |

All reagents were obtained from commercial suppliers and used without further purification unless otherwise stated.

### Synthesis of trans-1-Boc-4-fluoro-L-proline benzyl ester

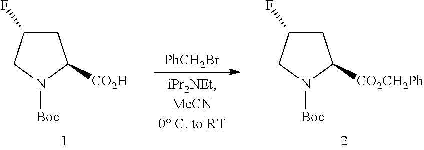

trans-1-Boc-4-fluoro-L-proline (1, 100 g; 428 mmol) was suspended in dry MeCN (450 ml) and stirred with a stir-bar at room temp in a 1 L flask under nitrogen. N,N-diisopropylethylamine (57.08 g/77 ml; 441.6 mmol) was added over 30 min from an addition funnel. The solid dissolved to form a pale-yellow solution at room temperature and the solution was cooled to 0-5° C. in an ice-water bath and stirred for 15 minutes. Benzyl bromide (75.53 g/52.5 ml) was added from an addition funnel into the yellow solution over 1 hour. The reaction was stirred overnight with the temperature rising from 0° C. to room temperature. Ester formation was complete (<2% 1 remaining), as judged by the LCMS data, after stirring ~24 hours. The mixture was concentrated at reduced pressure using a rotary evaporator connected to a Teflon pump (~30 mm vacuum) and the residue was partitioned with 2-methyl-tetrahydrofuran (2-MeTHF) (400 ml) and aqueous citric acid (50 g dissolved in 250 ml water). The phases were separated, and the aqueous layer was re-extracted with 2-MeTHF (2×100 ml). The 2-MeTHF layers were combined, washed with water (150 ml) and concentrated in vacuo. The off-white solid crude product was purified by crystallization from MTBE-hexanes (~1:4 v/v) with a 1^{st} crop yield of 2 of 113.1 g. The mother liquors concentrated and crystallized giving the 2^{nd} crop (yield 15.2 g). The total yield of the benzyl ester 2 was 128.3 g (92.5%, 98.2 LCAP purity).

¹H-NMR (CDCl₃, 400 MHz): δ 1.36 (s) and 1.48 (s) (9H, tBu); 2.10 (1H, complex, —CH—); 2.60 (1H, complex, —CH—); 3.61 (1H, complex —CH—N); 3.87 (ddd, 1H, —CH—N); 4.45 (t) and 4.54 (t) (1H, —CH—CO—); 5.09 (complex) and 5.28 (complex) (3H, —CH₂—O and —CH—F); 7.36 ppm (5H, complex, Ph).

¹⁹F-NMR (CDCl₃; 282 MHz): δ-177.56 (multiplet), −176.90 (multiplet).

LCMS: 346.1 (M+Na)+.

### Synthesis of trans-4-fluoro-L-proline benzyl ester

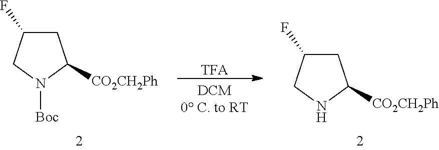

Trifluoroacetic acid (TFA) (25 ml) was combined with dry dichloromethane (25 ml) and stirred with a stir-bar at 0° C. in a 500 ml flask under nitrogen. trans-1-Boc-4-fluoro-L-proline benzyl ester (2, 20 g; 61.9 mmol) was added in ~2 g portions over 10 min. A pale-yellow solution formed. The cooling bath was removed, and the solution was allowed to come to room temperature. After 8 hours a small aliquot was removed and checked by LCMS and HPLC that showed the reaction was complete (2 not detected by HPLC). The mixture was concentrated at reduced pressure and the yellow liquid residue was partitioned in a 500 ml separatory funnel with tert-butyl methyl ether (MTBE) (200 ml) and washed with saturated aqueous Na₂CO₃ (100 ml) followed by addition of 1M NaOH (~200 ml) to make the aqueous layer pH 12. The phases were separated and the lower aqueous layer was re-extracted with MTBE (2×100 ml). The MTBE extracts were combined and washed with brine (150 ml). The combined MTBE extract was filtered and concentrated at reduced pressure. The crude product was purified on a silica gel plug (~100 g; under slight vacuum of ~500 mm Hg) eluted with 15% EtOAc-hexane (150 ml), 50% EtOAc-hexane (150 ml) and 75% EtOAc-hexane (300 ml). The filtrate from the 50% and 75% EtOAc-hexane eluents were combined and concentrated at reduced pressure to a slightly yellow liquid. The yield of trans-4-fluoro-L-proline benzyl ester 3 was 11.5 g (83.5%).

¹H-NMR (CDCl₃, 400 MHz): δ 2.00 (1H, multiplet, —CH); 2.13 (1H, multiplet, —CH); 2.45 (1H, ddd, NH); 3.17 (1H, dq, —CH—N); 3.22 (1H, q, —CH—N); 4.08 (1H, t, —CH—CO); 5.23 (1H, dt, —CHF), 5.17 (2H, s, —CH₂O—); 7.35 (5H, complex, Ph).

¹⁹F-NMR (CDCl₃; 282 MHz): δ-174.60 ppm.

LCMS: 224.1 (M+H)+.

### Synthesis of (5S,7R)-5-((benzyloxy)carbonyl)-7-fluoro-4-azaspiro[3.4]octan-4-ium triflate salt

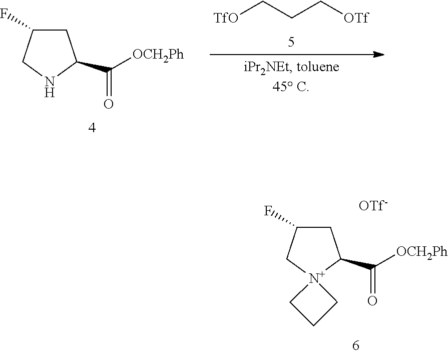

trans-4-Fluoro-L-proline benzyl ester (4, 2.0 g; 8.96 mmol) and N,N-diisopropylethylamine (3.12 ml; 17.92 mmol) were dissolved in dry toluene (65 ml) in a 100 ml flask equipped with a stir-bar under nitrogen. The toluene solution was heated and stirred in a heating bath to an internal temperature of ~45° C. A solution of 1,3-propanediol ditriflate (5, 3.04 g; 8.96 mmol) in toluene (10 ml) was added over 30 mins and then heating was continued for 4 h. The heating was discontinued, and the mixture allowed to cool to room temperature resulting in a biphasic mixture. The pale-yellow upper toluene layer was decanted off and saved and the lower layer gel remaining in the flask consisting of the product triflate salt 6 was slowly dried under a stream of nitrogen overnight. The amount of solid containing product and reagent related salts after drying overnight was 7.5 g.

LCMS: 264.1 (M)⁺ for amine salt molecular cation.

### Synthesis of benzyl (2R,7aS)-2-fluorotetrahydro-1H-pyrrolizine-7a(5H)-carboxylate

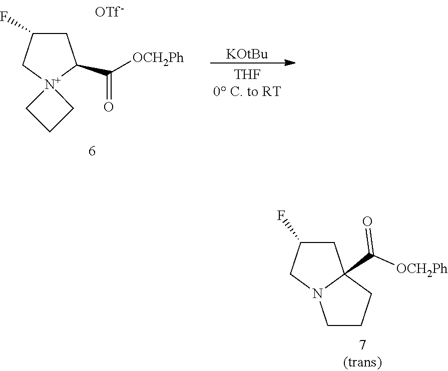

The crude (5S,7R)-5-((Benzyloxy)carbonyl)-7-fluoro-4-azaspiro[3.4]octan-4-ium triflate salt (6, 7.36 g, ca 8.96 mmol assuming quantitative conversion from 4) was dissolved in dry THF and stirred under nitrogen atmosphere at 0° C. (external ice/water bath temperature). A solution of potassium tert-butoxide (12.9 ml; 1M in THF, 12.9 mmol) was added over 15 mins via syringe to the solution of ammonium salt 6. The cold bath was removed, and the mixture stirred at room temperature for 2 hours. The reaction was worked up by filtering through a Celite pad and the pad flushed with THF (5 ml). The combined THF filtrates were concentrated at reduced pressure to give a beige-looking gel-powder. The sample was dissolved in DCM and chromatographed on a Biotage SP4 system using a silica gel cartridge (25 g) with a 0-100% EtOAc-hexanes gradient: crude ~1 g; 25 g silica column (Sfar-silica cartridge); eluent=100% Hex (3 column volumes (CV); eq.), 15% to 100% EtOAc-Hex (15 CV), 100% EtOAc (4 CV); detection 210 nm and 254 nm (collect all); threshold 35 mAU, test tube 16×150 mm; collecting 30×21 mL fractions; rich cut fractions #9-14 were combined and concentrated to an oil (322 mg, 13.6% from 4) that had benzyl (2R,7aS)-2-fluorotetrahydro-1H-pyrrolizine-7a(5H)-carboxylate (7).

¹H-NMR: (CDCl₃; 400 MHz): δ 1.80-1.97 (3H, complex, 3×—CH—); 2.25 (1H, dd, —CH—); 2.37-2.54 (2H, complex, —CH₂—); 2.93 ppm (1H, complex, —CH—N); 3.23-3.86 (3H, complex 3×—CH—N) 5.13 (2H, s, CH₂O) 5.21 (1H, broad d, —CH—F) and 7.34 (5H, complex, Ph).

¹⁹F-NMR (CDCl₃; 282 MHz): δ-173.34 ppm (complex).

LCMS: 264.1 (M+H)⁺.

### Synthesis of ((2R,7aS)-2-fluorotetrahydro-1H-pyrrolizin-7a(5H)-yl)methanol

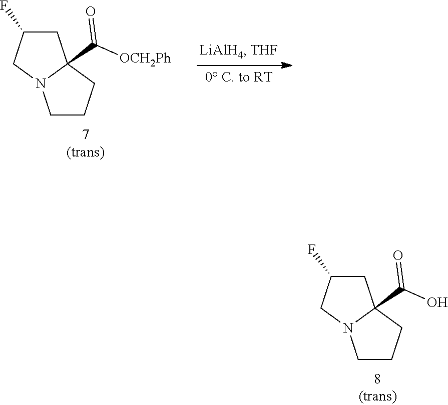

Benzyl (2R,7aS)-2-fluorotetrahydro-1H-pyrrolizine-7a(5H)-carboxylate (7, 263 mg; 1 mmol) was dissolved in dry THF (5 ml) and stirred with a stir-bar at 0° C. (external ice-water bath temperature) under a nitrogen atmosphere. A solution of lithium aluminum hydride (2 M in THF, 1.5 mL) was added using a syringe at 0° C. over 5 min. The cold bath was removed after the addition was completed and the reaction was stirred for 30 min at room temperature. The progress of the reaction was evaluated by LCMS, and there was no starting material detectable. The reaction was quenched by adding saturated aqueous Na₂SO₄ solution resulting in hydrogen gas evolution and formation of white gelatinous solids. The quenched mixture was filtered through a Celite plug and the plug washed with ~30 mL CH₂Cl₂ collecting all the filtrates, which were concentrated in vacuo to give a colorless oil (85 mg) of the ((2R,7aS)-2-fluorotetrahydro-1H-pyrrolizin-7a(5H)-yl)methanol 8 with a small amount of benzyl alcohol.

¹H-NMR: (CDCl₃; 400 MHz): δ 0.6-1.13 (6H, complex, 3×—CH₂—); 2.7 (1H, br, —OH); 2.77-3.2 (4H, complex, 2×—CH₂N—CH₂—)); 3.24 and 3.37 ppm (2H, ABq, —CH₂O); 5.11 and 5.30 ppm (1H, complex, CHF).

¹⁹F-NMR (CDCl₃; 282 MHz): δ-172.91 ppm (complex).

LCMS: 160.1 (M+H)⁺.

### Synthesis of 1,3-propanediol ditriflate

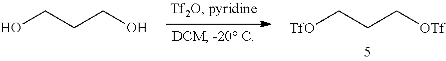 [SMILES: OCCCO ; O=S(=O)(OCCCOS(=O)(=O)C(F)(F)F)C(F)(F)F]

Before starting this experiment 1,3-propanediol was stored over molecular sieves 4A for a week to remove trace amounts of water.

1,3-Propanediol (8.75 g; 115 mmol) was dissolved in DCM (50 ml) in a dry 1 L flask with a stir bar under nitrogen and then pyridine (18.7 g/20.7 ml; 236 mmol) was added and the mixture cooled to 0° C. In a dry 250 ml flask, triflic anhydride (68.3 g/40 ml; 242 mmol) was added to DCM (125 ml) and stirred as a homogenous solution at −20° C. The solution of propanediol with pyridine was transferred to a 125 mL addition funnel and added to the triflate solution in 10 min. The reaction was stirred at −20° C. for 15 min then the flask was removed and shaken for 10 seconds. The reaction flask was placed back into the −20° C. cold bath and stirred for 0.5 h and then stirred for 1 h as the reaction warmed to room temperature as a pink solution with a white precipitate. The reaction was poured into water (250 ml), the layers were separated, and the organic layer washed with water (2×150 ml). The pale pink solution was dried over anhydrous Na₂SO₄ for ~10 min then filtered through a silica plug (80 g) collecting the filtrate. The silica plug was washed with fresh DCM (150 ml), filtered and the filtrates were combined. The volatiles were removed in vacuo by rotary evaporation to afford a pale pink liquid. The yield of 1,3-propanediol ditriflate was 35.5 g (90.5%). The ditriflate was stored at ~0° C.

¹H-NMR (CDCl₃; 400 MHz): δ 2.37 (2H, quin, —CH₂—); 4.80 ppm (4H, t, —CH₂OTf).

¹⁹F-NMR (CDCl₃; 282.2 MHz): δ-74.51 ppm.

What is claimed:

1. A method of preparing a compound of Formula (II-1): 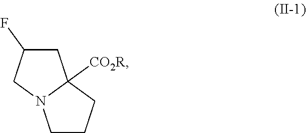
or a salt thereof, wherein the method comprises reacting a compound of Formula (III-1): 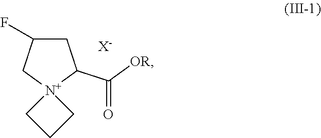
with a first base in the presence of a first solvent, thereby producing a compound of Formula (II-1) or a salt thereof, wherein R is an ester protecting group and X is a counter ion.

2. The method of claim 1, wherein the reaction of a compound of formula (III-1) with a first base is conducted at between about 0° C. to about room temperature.

3. The method of claim 1 or 2, wherein the first base is a metal alkoxide base.

4. The method of claim 3, wherein the first base is KOtBu.

5. The method of any one of claims 1-4, wherein the first solvent is a polar aprotic solvent.

6. The method of claim 5, wherein the polar aprotic solvent is an ethereal solvent.

7. The method of claim 5 or 6, wherein the polar aprotic solvent is THF or 2-MeTHF.

8. The method of any one of claims 1-7, wherein the method produces a compound of formula (II-1) as a diastereomerically pure compound.

9. The method of any one of claims 1-8, wherein the compound of Formula (II-1) is a compound of Formula (IIa-1): 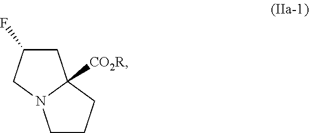
or a salt thereof, and the compound of Formula (III-1) is a compound of Formula (IIIa-1):

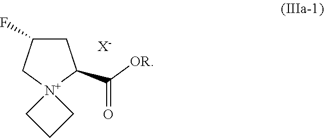

10. The method of any one of claims 1-9, wherein the method further comprises: reacting a compound of Formula (IV-1): 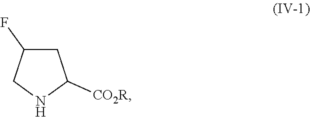
or a salt thereof, with an alkylating agent in the presence of a second base and a second solvent, thereby producing a compound of Formula (III-1):

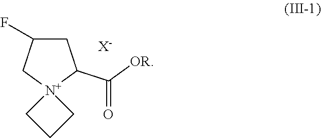

11. The method of claim 10, wherein the reaction of a compound of formula (IV-1) with an alkylating agent is conducted at about room temperature.

12. The method of any one of claims 1-11, wherein X⁻ is triflate (TfO⁻) ion.

13. The method of any one of claims 10-12, wherein the alkylating agent is 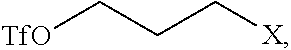
wherein X is triflate or halide.

14. The method of claim 13, wherein the alkylating agent is:

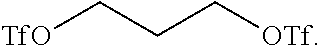 [SMILES: O=S(=O)(OCCCOS(=O)(=O)C(F)(F)F)C(F)(F)F]

15. The method of any one of claims 10-14, wherein the second base is an amine.

16. The method of any one of claims 10-14, wherein the second base is a weak base anion-exchange resin.

17. The method of claim 16, wherein the anion-exchange resin is Amberlyst™ A-21.

18. The method of any one of claims 15-17, wherein the amine is N,N-diisopropylethylamine.

19. The method of any one of claims 10-18, wherein the second solvent is a nonpolar solvent.

20. The method of any one of claims 10-19, wherein the second solvent is selected from the group consisting of toluene, THF, 2-Me THF, and acetonitrile.

21. The method of claim 20, wherein the second solvent is acetonitrile.

22. The method of any one of claims 10-21, wherein the compound of formula (IV-1) is a compound of Formula (IVa-1): 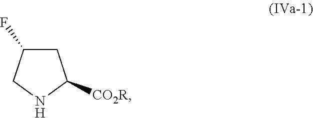
or a salt thereof.

23. The method of any one of claims 10-22, wherein the method further comprises reacting a compound of Formula (V-1): 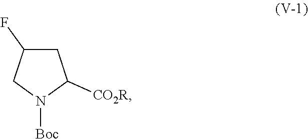
or a salt thereof, with an acid in the presence of a third solvent, thereby producing a compound of Formula (IV-1): 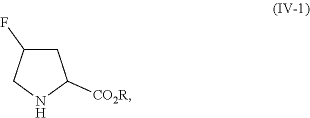
or a salt thereof.

24. The method of claim 23, wherein the acid is an organic acid or mineral acid.

25. The method of claim 24, wherein the acid is trifluoroacetic acid.

26. The method of any one of claims 23-25, wherein the third solvent is a polar aprotic solvent.

27. The method of any one of claims 23-26, wherein the third solvent is dichloromethane.

28. The method of any one of claims 23-27, wherein the compound of Formula (V-1) is a compound of Formula (Va-1): 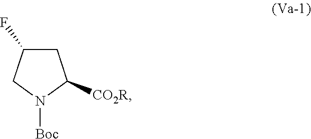
or a salt thereof.

29. The method of any one of claims 23-28, wherein the method further comprises reacting a compound of Formula (VI): 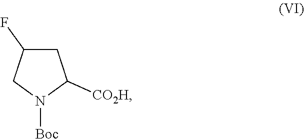 [SMILES: CC(C)(C)OC(=O)N1CC(F)CC1C(=O)O]
or a salt thereof, with an agent that introduces an R group in the presence of a third base and a fourth solvent, thereby producing a compound of Formula (V-1): 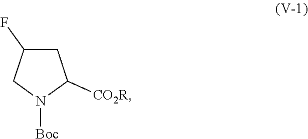
or a salt thereof.

30. The method of claim 29, wherein the third base is an amine base.

31. The method of claim 30, wherein the amine base is selected from the group consisting of N,N-diisopropylethylamine, triethylamine, and 1,8-diazabicyclo(5.4.0)undec-7-ene (DBU).

32. The method of any one of claims 29-31, wherein the fourth solvent is a polar aprotic solvent.

33. The method of claim 32, wherein the fourth solvent is acetonitrile.

34. The method of any one of claims 29-33, wherein the compound of Formula (VI) is a compound of Formula (VIa): 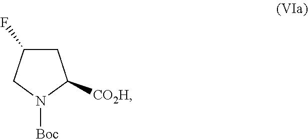 [SMILES: CC(C)(C)OC(=O)N1C[C@H](F)C[C@H]1C(=O)O]
or a salt thereof.

35. The method of any one of claims 29-34, wherein the agent that introduces an R group is a benzyl halide.

36. The method of claim 35, wherein the agent that introduces an R group is benzyl bromide.

37. The method of any one of claims 1-36, wherein R is benzyl.

38. A method of preparing a compound of Formula (I): 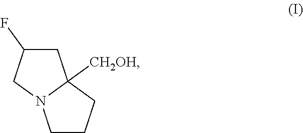 [SMILES: OCC12CCCN1CC(F)C2]
or a salt thereof, wherein the method comprises:
reacting a compound of Formula (II-1): 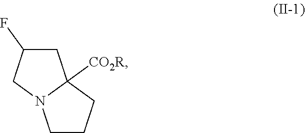
or a salt thereof with a reducing agent in the presence of a fifth solvent, thereby producing a compound of Formula (I) or a salt thereof, wherein the compound of Formula (II-1) or a salt thereof is prepared according to a method of any one of claims 1-37.

39. The method of claim 38, wherein the reducing agent is a metal hydride.

40. The method of claim 39, wherein the metal hydride is selected from the group consisting of LiAlH₄, LiBH₄, and Red-Al®.

41. The method of any one of claims 38-40, wherein the fifth solvent is a polar aprotic solvent.

42. The method of any one of claims 38-41, wherein the fifth solvent is an ethereal solvent.

43. The method of any one of claims 38-42, wherein the fifth solvent is selected from the group consisting of THF, Me-THF, diethyl ether, dimethoxy ethane, and 1,4-dioxane.

44. The method of any one of claims 38-43, wherein the method produces a compound of formula (I) as a diastereomerically pure compound.

45. The method of any one of claims 38-44, wherein the compound of Formula (I) is a compound of Formula (Ia): 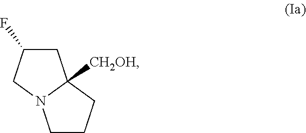 [SMILES: OC[C@@]12CCCN1C[C@H](F)C2]
or a salt thereof.

46. A method of preparing a compound of Formula (I): 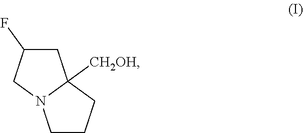 [SMILES: OCC12CCCN1CC(F)C2]
or a salt thereof, wherein the method comprises the steps of:
(1) reacting a compound of formula (VI): 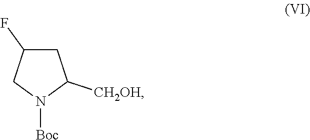 [SMILES: CC(C)(C)OC(=O)N1CC(F)CC1CO]
or a salt thereof with an agent that introduces an R group, thereby producing a compound of Formula (V-1): 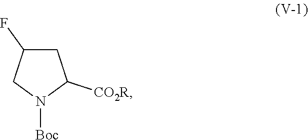
or a salt thereof;
(2) reacting a compound of Formula (V-1) or a salt thereof with an acid, thereby producing 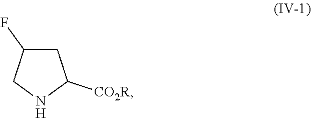
or a salt thereof;
(3) reacting a compound of Formula (IV-1) or a salt thereof with an alkylating agent, thereby producing a compound of Formula (III-1): 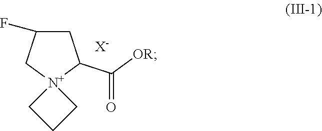
(4) reacting a compound of Formula (III-1) with a base, thereby producing a compound of Formula (II-1): 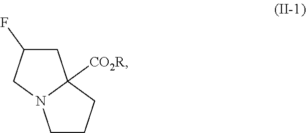
or a salt thereof; and
(5) reacting a compound of Formula (II-1) or a salt thereof with a reducing agent, thereby producing a compound of Formula (I) or a salt thereof;
wherein R is an ester protecting group and X is a counter ion.

47. The method of claim 46, wherein step (4) produces a compound of formula (II-1) as a diastereomerically pure compound.

48. The method of claim 46 or 47, wherein the compound of Formula (II-1) is a compound of Formula (IIa-1): 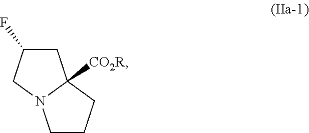
or a salt thereof, and the compound of Formula (III-1) is a compound of Formula (IIIa-1):

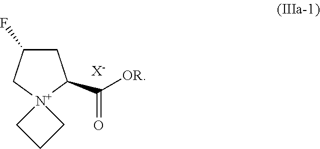

49. The method of any one of claims 46-48, wherein the compound of Formula (I) is a compound of Formula (Ia): 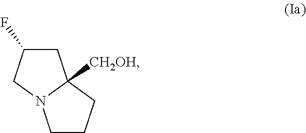 [SMILES: OC[C@@]12CCCN1C[C@H](F)C2]
or a salt thereof.

50. The method of any one of claims 46-49, wherein the compound of formula (IV-1) is a compound of Formula (IVa-1): 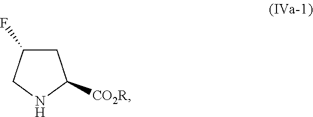
or a salt thereof.

51. The method of any one of claims 46-50, wherein the compound of Formula (V-1) is a compound of Formula (Va-1): 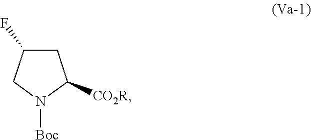
or a salt thereof.

52. The method of any one of claims 46-51, wherein the compound of Formula (VI) is a compound of Formula (VIa): 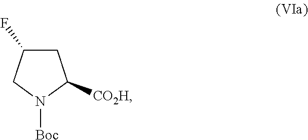 [SMILES: CC(C)(C)OC(=O)N1C[C@H](F)C[C@H]1C(=O)O]
or a salt thereof.

53. The method of any one of claims 46-52, wherein the reaction of step (4) is conducted at about 0° C. to about room temperature.

54. The method of any one of claims 46-53, wherein the reducing agent is a metal hydride.

55. The method of claim 54, wherein the metal hydride is selected from the group consisting of LiAlH₄, LiBH₄, and Red-Al®.

56. The method of claim 54, wherein the metal hydride is LiAlH₄.

57. The method of any one of claims 46-56, wherein the base is a metal alkoxide base.

58. The method of any one of claims 46-57, wherein the base is KOtBu.

59. The method of any one of claims 46-58, wherein the reaction of step (3) is conducted at room temperature.

60. The method of any one of claims 46-59, wherein X⁻ is triflate (TfO⁻) ion.

61. The method of any one of claims 46-60, wherein the alkylating agent is 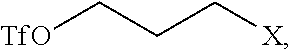
wherein X is triflate or halo.

62. The method of claim 61, wherein the alkylating agent is:

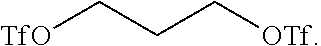 [SMILES: O=S(=O)(OCCCOS(=O)(=O)C(F)(F)F)C(F)(F)F]

63. The method of any one of claims 46-62, wherein the acid is an organic acid or mineral acid.

64. The method of claim 63, wherein the acid is trifluoroacetic acid.

65. The method of any one of claims 46-64, wherein R is benzyl.

66. The method of any one of claims 46-65, wherein the agent that introduces an R group is a benzyl halide.

67. The method of claim 66, wherein the agent that introduces an R group is benzyl bromide.

68. A compound of Formula (III): 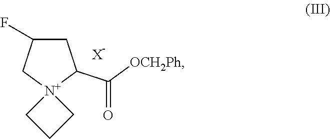
wherein X⁻ is a counter ion.

69. The compound of claim 68, wherein the compound is a compound of Formula (IIIa): 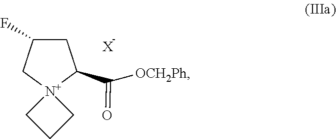
wherein X⁻ is a counter ion.

70. The compound of claim 68 or 69, wherein X⁻ is triflate (TfO⁻) ion.

71. A compound of Formula (II): 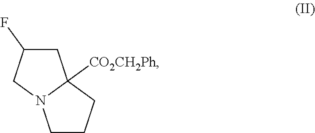
or a salt thereof.

72. The compound of claim 71, wherein the compound is a compound of Formula (IIa): 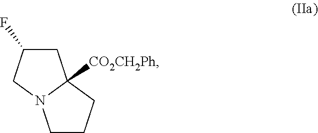
or a salt thereof.

73. A compound of Formula (II-1): 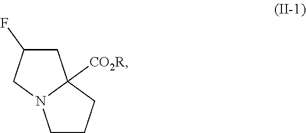
or a salt thereof, wherein R is an ester protecting group, wherein the compound is prepared according to a method of any one of claims 1-37.

74. A compound of Formula (I): 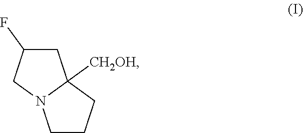 [SMILES: OCC12CCCN1CC(F)C2]
or a salt thereof, wherein the compound of Formula (I) is prepared according to a method of any one of claims 38-67.
